(19) United States
(12) Patent Application Publication
(10) Pub. No.: US 2010/0047272 A1
(43) Pub. Date: Feb. 25, 2010

(54) Therapeutic vaccine comprising mycobacterial heat shock protein 70

(75) Inventors: Adriaan Peter Koets, Utrecht (NL); Victor Pierre Marie Gerard Rutten, Utrecht (NL)

Correspondence Address: Intervet/Schering-Plough Animal Health, Patent Dept. K-6-1, 1990, 2000 Galloping Hill Road, Kenilworth, NJ, 07033-0530, US

(73) Assignee: Universiteit Utrecht Holding B.V., Ultrecht (NL)

(21) Appl. No.: 12/441,160

(22) PCT Filed: Oct. 1, 2007
(86) PCT No.: PCT/EP07/60351

§ 371 Date: Oct. 21, 2009

Related U.S. Application Data
(60) Provisional application No. 60/848,975, filed on Oct. 2, 2006.

(30) Foreign Application Priority Data
Oct. 2, 2006 (EP) 06121605.7

(51) Int. Cl. A61K 39/04; A61K 39/295; A61P 31/04; C12N 1/21; C12N 1/19; C12N 5/10

(52) U.S. Cl. 424/201.1; 424/248.1; 424/203.1; 435/252.3; 435/254.2; 435/325

(57) ABSTRACT

The present invention relates to the use of a mycobacterial heat-shock protein for the manufacture of a vaccine for therapeutic application.

The present invention relates to the use of a heat-shock protein for the manufacture of a vaccine for therapeutic application.

Many of the infectious diseases known today develop rapidly and, with or without the help of vaccines of pharmaceuticals, disappear equally rapidly. There exists however a group of micro-organisms that causes slow progressive infections that is hardly or not at all detectable in an early stage. This period of (real or purported) latency can last for years.

A prominent genus of micro-organisms that is notorious for causing slow progressive disease is the genus Mycobacterium, more specifically its species M. tuberculosis, M. avium, M. avium ssp. paratuberculosis and M. bovis.

M. tuberculosis is the cause of tuberculosis (tb) in i.a. humans, M. avium ssp. paratuberculosis causes paratuberculosis (paratb) in i.a. cattle and M. bovis is the cause of what can be seen as the bovine variant (bovine tb) of human tuberculosis.

These three Mycobacteria are very closely related, both with regard to their phenotype/genotype, and with respect to the nature of the slow progressive disease they cause.

Merely as an illustration of the close relatedness of these three species: the Mycobacterium bovis BCG vaccine is the only vaccine currently available for (albeit limited) protection against both human tb (caused by M. tuberculosis) and bovine tb (caused by M. bovis). This is illustrated by Vordermeier, H. M. et al., (Veterinary Journal 171: 229-244 (2006)) and by Dietrich, J. et al., (Tuberculosis 86: 163-168 (2006)).

Another aspect, or better; a consequence, of the close relatedness between the three Mycobacterium species is the established fact that they are infective to various mammalian species. Mycobacterium bovis tuberculosis in cats has been described, transmission of Mycobacterium bovis from animals, i.a. cats to humans has been described, transmission of 30 Mycobacterium tuberculosis from humans to animals, i.a. dogs has been described, the danger of feline tuberculosis for man has been described and so on. (Monies, B. et al., veterinary Record 158: 280 (2006), Monies, B. et al., veterinary Record 158: 245-246 (2006), Liu, S., Journ. Am. Vet Med. As. 177: 164-167 (1980), Snider, W., Am. Rev. Resp. Dis. 104: 877-887 (1971), Pavlik, I. et al., Veterinarni Medicina 50: 291-299 (2005), Baker, M. G. et al., Epidem & Inf. 134:1068-1073 (2006)).

Given the relatively low frequency of cross-species infection, it would not be useful or even sensible to consider prophylactic vaccination of all cats and dogs that are kept as pets. However a very attractive possibility would be a therapeutic vaccination in those cases in which an animal becomes infected by a Mycobacterial species. Such an approach would be much more attractive than the current treatment; the treatment with antibiotics, if only for the fact that currently many Mycobacterial species exist that are resistant to several antibiotics.

Such therapeutic vaccines are currently however not available.

Tuberculosis is a major health problem all over the world, and it is known to cause more than 2 million death annually. Treatment with antibiotics is efficacious and relatively easy, but is severely hampered by the weak socioeconomic structure of specifically those regions in the world where the disease is endemic. Therefore, an efficacious vaccine against the disease in humans is highly desired.

Bovine tuberculosis has a very high incidence in i.a. the UK, where it has been on the increase since 1988. It is also an increasing problem over the last decade in Northern Ireland. Bovine tb also remains an economic problem in countries with an endemic wildlife reservoir such as New Zealand and Ireland. Australia and most countries in Western Europe, on the other hand, have eradicated the disease. Nevertheless, the disease remains a problem in the developing countries, as a consequence of which 94% of the world's population lives in countries in which the control of bovine tb in cattle and/or buffaloes is non-existent.

Therefore, an efficacious vaccine against the disease in ruminants is also highly desired.

It is however known that the Mycobacterium bovis BCG vaccine, although it is the most widely used vaccine in the world, provides only limited protection against human tb and bovine tb. In the publications mentioned above, the problems encountered with the Mycobacterium bovis BCG vaccine are illustrated.

Paratuberculosis or Johne's disease (JD) in ruminants, is an infectious disease of the small intestine and considered a global problem of the livestock industry. The disease is caused by Mycobacterium avium spp. paratuberculosis (MAP), and leads to substantial economic losses (Johnson-Ifearulundu, Y., J. B. Kaneene, and J. W. Lloyd, J. Am. Vet. Med. Assoc, 1999. 214(6): p. 822-5). As it has been suggested that MAP is involved in the aetiology of Crohn's disease in humans, both animal and human health aspects justify further research into the immune pathogenesis of paratuberculosis aiming at control (if not eradication) of the disease by a combined strategy of the use of improved diagnostic tools and vaccination (Collins, M. T., J Dairy Sci, 1997. 80(12): p. 3445-8. Biet, F., M. L. Boschiroli, M. F. Thorel, and L. A. Guilloteau, Vet Res, 2005. 36(3): p. 411-36. Greenstein, R. J., Lancet Infect Dis, 2003. 3(8): p. 507-14. Chiodini, R. J. and C. A. Rossiter, Vet Clin North Am Food Anim Pract, 1996. 12(2): p. 457-67).

Young calves acquire the infection in the first months of life either through oral uptake of colostrum, milk or feces of infected cows or even before birth through infection via the intra-uterine route. They either successfully clear the infection, or become infected for life. The infected animals shed the bacteria in their feces and milk intermittently or continuously from an age of approximately 2 years onwards (Payne, J. and J. Rankin, Res. Vet. Sci., 1961. 2: p. 175-179. Payne, J. and J. Rankin, Res. Vet. Sci., 1961. 2: p. 167-174). After an incubation period of 4 to 5 years, a proportion of the infected animals shifts from the sub-clinical stage to the clinical stage and develops an incurable progressive form of protein losing enteropathy with chronic diarrhea resulting in death. Disease control and ultimately eradication has been attempted through various strategies such as test & cull, in which infected animals are removed from the herd, calf management, aimed at preventing infection of the most susceptible animals and to a lesser extend vaccination. As transmission of MAP does not only occur through the oral-fecal route but also through intra-uterine transmission some of the measures aimed at preventing infection of young calves can not be completely effective (Sweeney, R. W., Vet Clin North Am Food Anim Pract, 1996. 12(2): p. 305-12. Sweeney, R. W., R. H. Whitlock, and A. E. Rosenberger, Am J Vet Res, 1992. 53(4): p. 477-80. Seitz, S. E., L. E. Heider, W. D. Heuston, S. Bech-Nielsen, D. M. Rings, and L. Spangler, J Am Vet Med Assoc, 1989. 194(10): p. 1423-6).

Some 75 years ago, the classic experiments of Valleé and Rinjard demonstrated that vaccination could be used to slow down the onset of the clinical signs of the disease. This effect is most likely due to slower progression of the infection in affected animals.

The currently available vaccines are variations of whole bacterins with adjuvants. These vaccines have been shown to have a variable efficacy in field studies. (Kohler, H., et al., J Vet Med B Infect Dis Vet Public Health 2001, 48(3), 185-195. Muskens, J., van Zijderveld, F., Eger, A. & Bakker, D., Veterinary Microbiology 2002, 86(3), 269-278).

From these studies it has become apparent that that vaccination of calves in the first month of life with a whole cell type vaccine (inactivated) prevents to a certain degree the development of the clinical stage of the disease, and thus reduces economical damage. In cattle however, this type of vaccination does not result in elimination of mycobacteria since sub clinically infected animals were detected in approximately the same frequency in vaccinated herds and in non-vaccinated herds. Thus, the vaccine does not prevent infection and limits the frequency of sub clinically infected animals, which shed bacteria in their feces intermittently, only marginally at best.

In addition this vaccination strategy interferes with bovine tuberculosis diagnostics, as a consequence the use of inactivated whole cell vaccines is limited or even prohibited (e.g. in the Netherlands).

Finally, the inactivated whole cell vaccine also causes substantial tissue damage at the vaccination site and misuse of the vaccine in cattle as well as accidental self inoculation, e.g. by veterinarians, may have serious side-effects. These serious drawbacks, from an epidemiological, animal health and human health point of view, currently limit the use of vaccination of cattle worldwide and constitute a major problem in eradication of paratuberculosis.

Vaccination with a combination of subunits of mycobacterial subunits has been shown to have at least some effect in protection against the related Mycobacterium bovis. Skinner described prime-boost vaccinations with a vaccine on the basis of a combined Hsp65, Hsp70 and Apa DNA vaccine and BCG (Skinner, M. A. et al, Infection and Immunity, 2005, 73(7): 4441-4444). Such vaccines however require both priming and boosting with the protein-vaccine and the DNA-vaccine respectively, or vice versa, and in addition they require all three different antigens.

In US-Patent Application US 2003/0073094, the use of stress proteins of Mycobacterium tuberculosis has been described as a way of modulating an individual's immune response. In this application, stress proteins such as heat shock protein Hsp70 are used as a way of stimulating the immune system in a non-specific manner, specifically in prophylaxis and as a general and non-specific immune stimulant against cancer and auto-immune disease.

PCT-application WO 02/067982 describes whole-cell vaccines in which such stress-proteins are over-expressed. Such whole-cell vaccines would however not be the vaccine of choice since they interfere with bovine tuberculosis diagnostics, as described above.

Koets A. et al., have shown that a prophylactic vaccination with Mycobacterium avium spp. paratuberculosis Hsp70, although not protective, decreases the level of shedding of Mycobacterium avium spp. paratuberculosis in the feces during the first two years (Koets, A., et al., Vaccine 2006, 24: 2550-2559). In EP 1 510 821 the same authors suggested the use of Hsp70 as a therapeutic agent. However, no positive effect of this suggested therapeutic application was shown. Moreover, therapeutic application of Hsp70 after the patients have become shedders is not mentioned, nor suggested in this European patent application.

Shedding however becomes increasingly worse after the two year period, and apart from this the onset of the clinical stage also occurs only after two years, typically between 2 and 5 years.

As is clear from what has been said above, it is very difficult, if not impossible to avoid contamination of newborn calves. They may already become infected during birth.

This implies that a real prophylactic vaccination may not be possible.

About a decade ago, for the related bacterium Mycobacterium tuberculosis it has been suggested to try and vaccinate with a whole cell vaccine, not in a prophylactic way but instead in a therapeutic way: as a way of post-exposure vaccination (Fine, P. E., Novartis Foundation symposium 1998).

The results however were disappointing: Turner, J. et al., have unambiguously shown that such an approach does not at all modulate the course of an aerosol infection with Mycobacterium tuberculosis (Turner, J et al., Infection and Immunity 2000, 68(3): 1706-1709).

This is a strong indication that, at least for Mycobacterium tuberculosis that is closely related to Mycobacterium avium spp. paratuberculosis, this post-vaccination approach is useless.

Lowrie D. B. et al., have given a possible explanation for this phenomenon: they have shown that a type-2 cellular immune response, as is triggered by protein antigens is abundant during Mycobacterium tuberculosis infection but does not contribute to protection. A shift in the balance towards type-1 cellular immune responses might therefore be beneficial. This shift can be induced by using DNA vaccines instead of protein-based vaccines. And indeed they have shown that post-exposure vaccination with DNA vaccines comprising the 65 kD heat shock protein (Hsp65), contrary to vaccination with the protein itself provides a significant protection against the disease and indeed induces an immune response that even kills the bacteria. A much less significant protection was found for a DNA vaccine comprising Hsp70.

These findings are consistent with the findings of Skinner, M. A. as described above.

Therefore, for therapeutic or post-exposure vaccination, if feasible at all, vaccination with a DNA-vaccine, more specifically a DNA vaccine comprising Hsp65 seems to be the preferred vaccination approach.

It was now surprisingly found that post-infection vaccination with a vaccine comprising the 70 kD heat-shock protein (Hsp70), even when the infected mammals have become shedders, very significantly decreases the level of shedding. This is even true for heavily shedding animals. This is highly unexpected in view of the findings described by Lowrie and by Skinner as discussed above. It is even more surprising, since Hsp70 is a very immuno dominant protein that is known to induce a strong type-1 T-cell response already during infection, which does however not lead to any protection. Therefore, the appearance of a protective effect after post-infection, post-shedding development vaccination with Hsp70 as a single sub-unit could certainly not be expected.

Even more surprisingly, it was found that in animals suffering from the clinical stage of infection this stage can be stopped from deteriorating or can even improve, after therapeutic administration of a Hsp70 vaccine. This is indeed highly unexpected since progression from the prolonged asymptomatic stage to a stage of clinical signs of infection was always considered to be always irreversible and fatal.

The decrease in shedding is a mere parameter indicating that the progression of the disease can slowed down and possibly even stopped, even years after the moment of infection, i.e. when the disease has reached the moment at which it becomes visible through shedding. In the case of e.g. human tuberculosis, this moment would be comparable with the moment at which the infected human being, after years of latency, shows pathologic coughing and thus starts spreading the bacterium.

The bottom-line is that apparently, as reflected by the decrease in shedding, progression of the disease can be slowed down or even stopped, even years after the moment of infection, as a result of therapeutic vaccination.

These unexpected findings are equally applicable to M. tuberculosis, M. avium, M. avium ssp. paratuberculosis and M. bovis, due to their extremely close genotypic and phenotypic relationship, and the highly comparable progress of the disease they cause. The progression of bovine and human tuberculosis and tuberculosis in dogs, cats and other susceptible animals can equally be slowed down and eventually stopped.

Thus, a first embodiment of the present invention relates to the use of an immunologically active amount of mycobacterial Hsp70 protein or an immunogenic fragment thereof, for the manufacture of a vaccine for therapeutic application in mammals infected with bacteria of the genus Mycobacterium and which mammals have started shedding these bacteria. Shedding these bacteria in the sense of the present invention means shedding of bacteria of the same genus in the feces of the mammal after multiplication of the pathogenic bacteria after they gained access to their host tissue (i.e. after colonization).

Preferably, the mammals are humans, dogs, cats or ruminants.

Therapeutic use is the use of the vaccine as a therapeutic vaccine, contrary to the standard, i.e. prophylactic, use of a vaccine. Prophylactic use is the use before infection takes place, or around the moment of infection.

Therapeutic use for the purpose of this application is considered to be the use of the vaccine according to the invention after a first exposure to infection. This holds true for infections in animals and in humans. Next to this, the vaccine is used in humans or animals that have started shedding.

In practice, in the case of infection in humans, dogs and cats such therapeutic use would start at the moment shedding becomes evident, which would usually only happen 3 months, more likely 6 months, more likely 12 months or later after infection.

An immunogenic dose is known in the art as the amount of immunogenic material that is, possibly in combination with an adjuvant, sufficient to trigger an immune response in the target mammalian species.

The concept of vaccination with an immunogenic fragment of Hsp70 instead of the whole Hsp70 protein is given further below.

Since nucleic acid sequences encoding the Hsp70 protein according to the present invention are known in the art, see below, it is now feasible to obtain this protein in sufficient quantities. This can e.g. be done by using expression systems to express the whole or parts of the gene encoding the Hsp70 protein. An essential requirement for the expression of the nucleic acid sequence is an adequate promoter functionally linked to the nucleic acid sequence, so that the nucleic acid sequence is under the control of the promoter. It is obvious to those skilled in the art that the choice of a promoter extends to any eukaryotic, prokaryotic or viral promoter capable of directing gene transcription in cells used as host cells for protein expression.

Functionally linked promoters are promoters that are capable of controlling the transcription of the nucleic acid sequences to which they are linked.

Constructs comprising the nucleic acid sequences encoding the Hsp70 protein under the control of a functionally linked promoter will be further referred to as recombinant DNA molecules. Such a promoter can be the native promoter of the protein gene or another promoter, provided that that promoter is functional in the cell used for expression. It can also be a heterologous promoter. When the host cells are bacteria, useful expression control sequences which may be used include the Trp promoter and operator (Goeddel, et al., Nucl. Acids Res., 8, 4057, 1980); the lac promoter and operator (Chang, et al., Nature, 275, 615, 1978); the outer membrane protein promoter (Nakamura, K. and Inouge, M., EMBO J., 1, 771-775, 1982); the bacteriophage lambda promoters and operators (Remaut, E. et al., Nucl. Acids Res., 11, 4677-4688, 1983); the α-amylase (B. subtilis) promoter and operator, termination sequences and other expression enhancement and control sequences compatible with the selected host cell.

When the host cell is yeast, useful expression control sequences include, e.g., α-mating factor. For insect cells the polyhedrin or p10 promoters of baculoviruses can be used (Smith, G. E. et al., Mol. Cell. Biol. 3, 2156-65, 1983). When the host cell is of vertebrate origin illustrative useful expression control sequences include the (human) cytomegalovirus immediate early promoter (Seed, B. et al., Nature 329, 840-842, 1987; Fynan, E. F. et al., PNAS 90 11478-11482, 1993; Ulmer, J. B. et al., Science 259, 1745-1748, 1993), Rous sarcoma virus LTR(RSV, Gorman, C. M. et al., PNAS 79, 6777-6781, 1982; Fynan et al., supra; Ulmer et al., supra), the MPSV LTR (Stacey et al., J. Virology 50, 725-732, 1984), SV40 immediate early promoter (Sprague J. et al., J. Virology 45, 773, 1983), the SV-40 promoter (Berman, P. W. et al., Science, 222, 524-527, 1983), the metallothionein promoter (Brinster, R. L. et al., Nature 296, 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992). The regulatory sequences may also include terminator and poly-adenylation sequences. Amongst the sequences that can be used are the well known bovine growth hormone poly-adenylation sequence, the SV40 poly-adenylation sequence, the human cytomegalovirus (hCMV) terminator and poly-adenylation sequences.

Bacterial, yeast, fungal, insect and vertebrate cell expression systems are very frequently used systems. Such systems are well-known in the art and generally available, e.g. commercially through Clontech Laboratories, Inc. 4030 Fabian Way, Palo Alto, Calif. 94303-4607, USA. Next to these expression systems, parasite-based expression systems are attractive expression systems. Such systems are e.g. described in the French Patent Application with Publication number 2 714 074, and in US NTIS Publication Ser. No. 08/043,109 (Hoffman, S, and Rogers, W.: Public. Date 1 Dec. 1993).

The use of a Hsp70 protein that originates from Mycobacterium avium, M. bovis or M. tuberculosis is preferred.

Thus, a preferred form of this embodiment of the present invention relates to the use of an immunologically active amount of mycobacterial Hsp70 protein or an immunogenic fragment thereof, for the manufacture of a vaccine for therapeutic application in mammals, wherein the Hsp70 protein is a Mycobacterium avium, M. bovis or M. tuberculosis Hsp70.

More preferred for use in a vaccine for the protection of mammals, preferably ruminants against paratuberculosis is the use of a Hsp70 protein that originates from Mycobacterium avium ssp. paratuberculosis.

Therefore, a more preferred form of this embodiment of the present invention relates to the use of an immunologically active amount of mycobacterial Hsp70 protein or an immunogenic fragment thereof, for the manufacture of a vaccine for therapeutic application in mammals, preferably ruminants, wherein the Hsp70 protein is a Mycobacterium avium ssp. paratuberculosis Hsp70.

More preferred for use in a vaccine for the protection of mammals, preferably ruminants, dogs or cats against M. bovis infection is the use of a Hsp70 protein that originates from Mycobacterium bovis.

More preferred for use in a vaccine for the protection of mammals, preferably humans, dogs or cats against M. tuberculosis infection is the use of a Hsp70 protein that originates from Mycobacterium tuberculosis.

Mycobacterial Hsp70 sequences are known in the art, and can be found i.a. under the following references:

Mycobacterium leprae: M95576
Mycobacterium tuberculosis: P0A5B9.
Mycobacterium bovis: P0A5C0
Mycobacterium avium spp. paratuberculosis: AF254578

When a protein is used for e.g. vaccination purposes or for raising antibodies, it is not necessary to use the whole protein. It is also possible to use a fragment of that protein that is capable, as such or coupled to a carrier such as e.g. KLH, of inducing an immune response against that protein, a so-called immunogenic fragment. An “immunogenic fragment” is understood to be a fragment of the full-length protein that still has retained its capability to induce an immune response in the host, i.e. comprises a B- or T-cell epitope. At this moment, a variety of techniques is available to easily identify DNA fragments encoding antigenic fragments (determinants). The method described by Geysen et al (Patent Application WO 84/03564, Patent Application WO 86/06487, U.S. Pat. No. 4,833,092, Proc. Natl. Acad. Sci. 81: 3998-4002 (1984), J. Imm. Meth. 102, 259-274 (1987), the so-called PEPSCAN method is an easy to perform, quick and well-established method for the detection of epitopes; the immunologically important regions of the protein. The method is used world-wide and as such well-known to man skilled in the art. This (empirical) method is especially suitable for the detection of B-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and U.S. Patent application NTIS U.S. Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzowsky for HIV-epitopes; The FASEB Journal 5:2412-2418 (1991).

Another very attractive approach for vaccination against mycobacterial infection is by using Live Recombinant Carriers (LRCs) comprising a gene or a fragment thereof encoding mycobacterial Hsp70 or an immunogenic fragment thereof, together with a pharmaceutically acceptable carrier. These LRCs are micro-organisms or viruses in which additional genetic information, in this case a gene or a fragment thereof encoding mycobacterial Hsp70 or an immunogenic fragment thereof, has been cloned. Animals infected with such LRCs will produce an immunogenic response not only against the immunogens of the carrier, but also against the immunogenic parts of the protein(s) for which the genetic code is additionally cloned into the LRC, e.g. Hsp70.

As an example of bacterial LRCs, attenuated Salmonella strains known in the art can attractively be used.

Live recombinant carrier parasites have i.a. been described by Vermeulen, A. N. (Int. Joum. Parasitol. 28: 1121-1130 (1998))

Also, LRC viruses may be used as a way of transporting the nucleic acid into a target cell. Live recombinant carrier viruses are also called vector viruses. Viruses often used as vectors are Vaccinia viruses (Panicali et al; Proc. Natl. Acad. Sci. USA, 79: 4927 (1982), Herpesviruses (E.P.A. 0473210A2), and Retroviruses (Valerio, D. et al; in Baum, S. J., Dicke, K. A., Lotzova, E. and Pluznik, D. H. (Eds.), Experimental Haematology today—1988. Springer Verlag, New York: pp. 92-99 (1989)).

The technique of in vivo homologous recombination, well-known in the art, can be used to introduce a recombinant nucleic acid into the genome of a bacterium, parasite or virus of choice, capable of inducing expression of the inserted nucleic acid according to the invention in the host animal.

Thus, still another embodiment of the present invention relates to the use of a live recombinant carrier encoding mycobacterial Hsp70 or an immunogenic fragment thereof, under the control of a functionally linked promoter for the manufacture of a vaccine for therapeutic application in mammals, in particular humans or ruminants, infected with bacteria of the genus Mycobacterium and which mammals have started shedding these bacteria.

It is clear that host cells comprising a gene or a fragment thereof encoding mycobacterial Hsp70 or an immunogenic fragment thereof under the control of a functionally linked promoter can be used for the production of Hsp70. There is no need to first extract Hsp70 from the host cell before using it as a vaccine: the host cell can also be used as such. The same is true in case a host cell comprises an LRC expressing Hsp70. Examples thereof are eukaryotic cells comprising a viral or bacterial LRC.

Thus, still another embodiment of the present invention relates to the use of a host cell comprising a gene or a fragment thereof encoding mycobacterial Hsp70 or an immunogenic fragment thereof, under the control of a functionally linked promoter for the manufacture of a vaccine for therapeutic application in mammals, in particular humans or ruminants, infected with bacteria of the genus Mycobacterium and which mammals have started shedding these bacteria.

This embodiment also relates to a host cell containing a live recombinant carrier comprising a gene or a fragment thereof encoding mycobacterial Hsp70 or an immunogenic fragment thereof under the control of a functionally linked promoter.

A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, bovine cells and cell lines, Chinese Hamster Ovary cells (CHO) or Crandell Feline Kidney-cells, also with appropriate vectors or recombinant viruses.

Vaccines based upon live recombinant carriers as described above, capable of expressing Hsp70 or immunogenic fragments thereof e.g. based upon a Salmonella carrier or a viral carrier have the advantage over subunit vaccines that they better mimic the natural way of infection of mycobacteria. Moreover, their self-propagation is an advantage since only low amounts of the recombinant carrier are necessary for immunization.

Especially, but not only, if the therapeutic vaccine manufactured according to the invention is administered to young animals, it would be beneficial to administer at the same time a vaccine against another virus or micro-organism.

A preferred form of such a combination vaccine is a vaccine comprising, in addition to Hsp70 or an immunogenic fragment thereof, another mammalian, preferably human-, cat-, dog- or ruminant-pathogenic virus or micro-organism, antigenic material of that virus or microorganism or genetic information encoding that antigenic material. Such a combination vaccine would induce protection against not only the detrimental effects of Mycobacterium bovis, tuberculosis, avium or avium spp. paratuberculosis, but also against other pathogens.

In case a vaccine for the protection against a bovine pathogenic Mycobacterial species is made, such ruminant-pathogenic micro-organisms or viruses are preferably selected from the group of Bovine Herpesvirus, bovine Viral Diarrhoea virus, Parainfluenza type 3 virus, Bovine Paramyxovirus, Foot and Mouth Disease virus, Bovine Respiratory Syncytial Virus, porcine circo virus, porcine respiratory reproductive syndrome virus, another Mycobacterium spp, a Mycoplasma spp., Pasteurella haemolytica, Staphylococcus aureus, Escherichia coli, Leptospira spp., Staphylococcus uberis, Theileria parva, Theileria annulata, Babesia bovis, Babesia bigemina, Babesia major, Trypanosoma species, Anaplasma marginale, Anaplasma centrale and Neospora caninum.

Vaccines according to the present invention may in a preferred presentation also contain an adjuvant. Adjuvants in general comprise substances that boost the immune response of the host in a non-specific manner. A number of different adjuvants are known in the art. Examples of adjuvants are Freund's Complete and Incomplete adjuvant, vitamin E, non-ionic block polymers, muramyldipeptides, Quill A®, mineral oil e.g. Bayol® or Markol®, vegetable oil, and Carbopol® (a homopolymer), or Diluvac® Forte.

The vaccine may also comprise a so-called “vehicle”. A vehicle is a compound to which the polypeptide adheres, without being covalently bound to it. Often used vehicle compounds are e.g. aluminum hydroxide, -phosphate or -oxide, silica, Kaolin, and Bentonite.

A special form of such a vehicle, in which the antigen is partially embedded in the vehicle, is the so-called ISCOM (EP 109.942, EP 180.564, EP 242.380)

More preferably, the adjuvant is an adjuvant that induces a Type-1 response, or shifts the type-1/type-2 balance towards a type-1 response. Adjuvant modulation of immune responses is i.a. described by Lindblad, E. et al., (Infection and Immunity 1997, 65: 623-629).

Even more preferably, the adjuvant is dimethyl dioctadecyl ammoniumbromide (DDA)

In addition, the vaccine may comprise one or more suitable surface-active compounds or emulsifiers, e.g. Span or Tween.

Often, the vaccine is mixed with stabilisers, e.g. to protect degradation-prone polypeptides from being degraded, to enhance the shelf-life of the vaccine, or to improve freeze-drying efficiency. Freeze-drying is a preferred method to significantly improve shelf-life and to avoid low-temperature storage. Useful stabilisers are i.a. SPGA (Bovamik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

In addition, the vaccine may be suspended in a physiologically acceptable diluent.

It goes without saying, that other ways of adjuvating, adding vehicle compounds or diluents, emulsifying or stabilizing a polypeptide are also embodied in the present invention.

Vaccines according to the invention can very suitably be administered in amounts ranging between 1 and 200 micrograms of proteins, although smaller doses can in principle be used. A dose exceeding 200 micrograms will, although immunologically very suitable, be less attractive for commercial reasons.

Vaccines based upon live attenuated recombinant carriers, such as the LRC-viruses and bacteria described above can be administered in much lower doses, because they multiply themselves during the infection. Therefore, very suitable amounts would range between 10³ and 10⁹ CFU/PFU for respectively bacteria and viruses.

Many ways of administration can be applied. Oral application is a very attractive way of administration, because it is not labor-intensive. A preferred way of oral administration is the packaging of the vaccine in capsules, known and frequently used in the art, that only disintegrate after they have passed the highly acidic environment of the stomach. Also, the vaccine could be mixed with compounds known in the art for temporarily enhancing the pH of the stomach

Systemic application is also suitable, e.g. by intramuscular application of the vaccine. If this route is followed, standard procedures known in the art for systemic application are well-suited.

A preferred route is the subcutaneous administration.

Vaccines based upon mycobacterial Hsp70 are also very suitable as marker vaccines. A marker vaccine is a vaccine that allows to discriminate between vaccinated and field-infected animals e.g. on the basis of a characteristic antibody panel, different from the antibody panel induced by wild type infection.

A vaccine based upon purified mycobacterial Hsp70 would only induce antibodies against that protein, whereas a vaccine based upon a live wild-type, live attenuated or inactivated whole Mycobacterium, as well as field infection would induce antibodies against many of the mycobacterial proteins: this would clearly give a highly different antibody panel.

A simple ELISA test, having wells comprising purified mycobacterial Hsp70 and wells comprising another mycobacterial protein suffices to test serum from animals and to tell if the animals are either vaccinated with a vaccine according to the invention or suffered from mycobacterial field infection; animals vaccinated with a vaccine comprising purified mycobacterial Hsp70 would not have antibodies against other mycobacterial proteins than the mycobacterial Hsp70. Animals that have been vaccinated with a live attenuated vaccine or have encountered a field infection with Mycobacterium would however have antibodies against all immunogenic Mycobacterium proteins and thus also against other, non-mycobacterial Hsp70 protein.

Suitable other mycobacterial proteins in the test described above are e.g. Hsp65 and Apa.

Thus, another embodiment of the present invention relates to a diagnostic test for the discrimination between vaccination with a vaccine according to the invention on the one hand and vaccination with a whole cell vaccine or a field infection on the other hand, wherein such a test comprises purified mycobacterial Hsp70 or an immunogenic fragment thereof and separately another, non-Hsp70 protein.

### EXAMPLES

### Example 1

### 2. Materials & Methods

### 2.1 Animals and Experimental Design

A total of 28 newborn calves (16 male and 12 female) were used in the current study. The calves were raised using conventional procedures and feed, and were checked daily for general health. The calves were randomly assigned one of three group housing pens for 9 or 10 calves each. Blood and fecal samples were taken every 6 weeks. Heparinized blood samples were used for isolation of lymphocytes, and serum samples were taken for serological analysis. Body weight was recorded on the same time points as blood samples were taken. Fecal samples were taken 13 times during the experiment, at days 0, 42, 91, 126, 168, 210, 252, 294, 336, 378, 462, 504, and 561 post infection.

### 2.2 Infection of Calves

All calves were infected orally using feces from a MAP infected cow which was characterized as a consistent shedder by fecal culture of the mycobactin-J dependant and IS900 PCR positive MAP. The calves received 9 dosis of 20 grams of feces, mixed with 100 ml milk replacer per dose by gavage feeding, during the first 21 days of the experiment at regular intervals. Semi-quantitative fecal culture indicated that >100 cfu/gr of feces were present in the inoculum. Hence, calves received a minimum total dose of 1.8×10⁴ cfU each.

### 2.3 Immunization of Calves

All calves were immunized once at the day 217 of the experiment. The immunization consisted of the administration of 200 μg of recombinant M. a. paratuberculosis Hsp70 in 1 ml phosphate buffered saline (PBS) containing 20 mg/ml dimethyl dioctadecyl ammonium bromide (DDA) adjuvant (Sigma Aldrich, USA), subcutaneously in the dewlap. Recombinant M. a. paratuberculosis Hsp70 was produced as published previously (Koets, A. P., Rutten, V. P., de Boer, M., Bakker, D., Valentin-Weigand, P. & van Eden, W. Infect Immun 2001, 69(3), 1492-1498).

### 2.4 Fecal Culture of MAP

Diagnosis of paratuberculosis infection was performed using the routine fecal culture system, based on published methods (Jorgensen, J. B., Acta Vet Scand 1982, 23(3), 325-335), at Veterinary Health Service, Deventer,

The Netherlands. Samples were checked for bacterial growth by colony count every 4 weeks, the first observation at 8 weeks post inoculation, and considered negative if after a culture period of 16 weeks no bacterial growth was observed. Bacterial growth was confirmed to be M. avium ssp. paratuberculosis based on mycobactin J dependence of the culture and the confirmation of the presence of the specific IS900 insertion sequence by PCR (Vary, P. H., Andersen, P. R., Green, E., Hermon-Taylor, J. & McFadden, J. J., J Clin Microbiol 1990, 28(5), 933-937). Results of fecal culture were scored semi-quantitatively between 0 and 9 based on the combination of time to positive (TTP), respectively 8, 12 or 16 weeks and the number of colonies counted (CFU) per gram of feces, as outlined in table 1.

TABLE 1
Semi-quantitative scoring of fecal culture results: cfu count
(bac followed by range of colony forming units (cfu) in the
sample) × time to positive culture (TTP) in weeks

| | TTP | 16 | 12 | 8 |
|---|---|---|---|---|
| CFU | bac neg | 0 | 0 | 0 |
| | bac 1-10 | 1 | 2 | 3 |
| | bac 10-100 | 4 | 5 | 6 |
| | bac 100+ | 7 | 8 | 9 |

fecal culture score (count × time to positive)

### 2.5 Comparative Tuberculin Skin Test

A comparative tuberculin skin test was conducted 8 weeks post vaccination. According to the official EU guideline (L179, 9.7.2002) 0.1 ml bovine tuberculin (2500 IU) and 0.1 ml avian tuberculin (2500 IU) (prepared according to EU and OIE regulations by Central Institute for Animal Disease Control (CIDC), Lelystad, The Netherlands) were applied intracutaneously in the neck of each animal. At 72 hours post tuberculin skin test, the skin-fold thickness was measured and corrected for skin-fold thickness at time of application. A reaction was recorded as positive when a positive bovine reaction was more than 4 mm greater when compared to the avian reaction. A reaction was recorded as dubious when a positive bovine reaction was between 1 and 4 mm greater when compared to the avian reaction. A negative reaction was recorded when a positive or inconclusive bovine reaction which was equal to or less than a positive or inconclusive avian reaction.

### 2.6 Isolation of Peripheral Blood Mononuclear Cells

Peripheral blood mononuclear cells (PBMC) were isolated from aseptically taken, heparinized blood samples using density gradient centrifugation, and cultured as published previously (Koets, A. P., Rutten, V. P., Hoek, A. et al., Vet Immunol Immunopathol 1999, 70(1-2), 105-115).

### 2.7 Antigens

Recombinant M. a. paratuberculosis Hsp 65 kD and Hsp 70 kD were produced according to methods described in detail earlier (Koets, A. P., Rutten, V. P., de Boer, M., Bakker, D., Valentin-Weigand, P. & van Eden, W., Infect Immun 2001, 69(3), 1492-1498, Colston, A., McConnell, I. & Bujdoso, R., Microbiology 1994, 140, 3329-3336). Purity of the recombinant Hsp65 and Hsp70 was checked using SDS-PAGE and preparations were tested for LPS contamination by Limulus assay (Sigma, St. Louis, USA).

The Hsp70 N-terminus protein fragment was produced by removing the C-terminus ‘protein binding domain’ through enzymatic digestion using the restriction endonucleases AflII (NE BioLabs) en HindIII (Gibco-Invitrogen) from the original pTrcH is C-Hsp70 expression plasmid (5 units of enzyme per μg DNA). The fragments were separated using agarose gel electrophoresis, the large fragment was isolated from the gel using the QIAEXII gel extraction kit (Qiagen). The restriction sites were blunted by using T4 polymerase and DNA was isolated using a DNA cleaning kit (Zymo Research). Subsequently, the vector DNA was ligated using the T4 DNA ligase containing Quick Ligation Kit (NE Biolabs) according to instructions provided by the manufacturer. The vector was used to transform E. coli DH5α (Library Efficiency DH5α Competent Cells, Life Technologies) according to instructions provided by the manufacturer. Induction of protein expression and protein purification were performed similar to the method described for the wild-type Hsp70 protein. The deletion mutant protein, termed RBS70, contained the first 357 amino acids of the 623 amino acids containing wild type Hsp70 protein, which was verified by plasmid DNA sequencing, PAGE and western blotting.

Purified protein deviate was prepared from M. a. paratuberculosis strain 3+5/C culture supernatant (PPD-P) and M. a. avium strain D4 culture supernatant (PPD-A) according to the OIE manual (Gilmour, N. J. L. & Wood, G. W. Paratuberculosis (Johne's Disease). In OIE Manual of Standards for Diagnostic Tests and Vaccines Office International des Epizooties, Paris, 1996. 218-228) at the Central Institute for Animal Disease Control (CIDC), Lelystad, The Netherlands). M. a. paratuberculosis strain 316F and M. a. avium strain D4 were grown at the Institute for Animal Health and Science (Lelystad, The Netherlands). E. coli strain DH5α was grown overnight in Luria Bertani (LB) medium at 37° C.

Concanavalin A was used as a positive control (2.5 μg/ml) and medium alone as a negative control.

### 2.8 Elispot Assay for Bovine IFNγ Secreting Cells

The Elispot assay for bovine IFNγ secreting cells was performed as published previously (Koets, A., Hoek, A., Langelaar, M. et al., Vaccine 2006, 24(14), 2550-2559). Spots were counted, and total spot area was calculated using an automated Elispot reader according to instructions provided by the manufacturer (A.EL.VIS GmbH, Hanover, Germany). Spot counting results were expressed as delta-spot forming cells (dSFC), calculated by subtracting the number of spots in medium control wells from the number of spots in antigen stimulated wells, unless stated otherwise. Medium alone was used as a negative control, concanavalin A (2.5 μg/ml) was used as positive control. PPD-P, PPD-A, Hsp70 and Hsp65 were used at a predetermined optimal concentration of 10 μg/ml. M. a. paratuberculosis strain 316F, M. a. avium strain D4, and E. coli DH5α were used at MOI 1:1 with the PBMC. All tests were performed in triplicate.

### 2.9 Lymphocyte Stimulation Tests

Lymphocyte Stimulation Tests (LST) were performed as described in detail earlier (Koets, A., Rutten, V., Hoek, A. et al. Progressive bovine paratuberculosis is associated with local loss of CD4(+) T cells, increased frequency of gamma delta T cells, and related changes in T-cell function. Infect Immun 2002, 70(7), 3856-3864) in 96 well microtitre plates (Costar, Cambridge, Mass., USA). In short, 100 μl of the density gradient isolated PBMC suspension (2.10⁶ cells/ml) and 100 μl of antigen per well, all tests were performed in triplicate. The mycobacterial antigens PPD-P, Hsp65, and Hsp70 were used in predetermined optimal concentrations of 10 μg/ml each. Strain 316F bacteria were briefly sonicated, counted and used in a concentration of 1.10⁷ CFU/ml. Concanavalin A was used as a positive control (2.5 μg/ml) and medium alone as a negative control.

Cells were cultured at 37° C. and 5% CO₂ in a humidified incubator for 3 days. Then 0.4 μCu ³H thymidine (Amersham International) was added to each well and cells were cultured for an additional 18 hrs. Subsequently, cells were harvested onto glass fibre filters and incorporation of ³H thymidine was measured by liquid scintillation counting and expressed as Stimulation Index (S.I.) which was calculated by dividing the cpm of a specific stimulation by the cpm of the medium control.

### 2.10 Serology of Calves

Serological responses to recombinant M. a. paratuberculosis Hsp70 protein were measured using a previously described ELISA technique ((Koets, A. P., Rutten, V. P., de Boer, M., Bakker, D., Valentin-Weigand, P. & van Eden, W., Infect Immun 2001, 69(3), 1492-1498), with minor modifications. All sera were diluted 10 times in blocking buffer and 100 μl was measured in duplicate. In addition, in each plate a positive and a negative control sample were added in duplicate. The modifications consisted of the use of 1 μg/ml isotype specific secondary antigens (mouse-anti-bovine IgG1, IgG2, IgA and IgM (Cedi-Diagnostics, Lelystad, Netherlands)) followed by 3 washes and incubation with 1 μg/ml peroxidase conjugated polyclonal goat-anti-mouse antibody (Nordic Laboratories, The Netherlands). Finally plates were washed 3 times and 100 μl ABTS substrate buffer (Boehringer Mannheim, Mannheim, Germany) was used to develop a color reaction which was read on an ELISA reader (Biorad) at 405 nm. Results are expressed as S/N (sample to negative) ratio.

### 3. Results

### 3.1 Observations on General Health Status

The growth of the animals was monitored throughout the experiment and the results indicated that calves grew comparable to conventionally reared unvaccinated calves. (data not shown) One animal was culled following a severe respiratory infection that was non-responsive to treatment.

### 3.2 Side Effects of Vaccination

The effect of the single vaccination with Hsp70 with DDA adjuvant in the dewlap was a palpable swelling with an average diameter of 2.6 cm±0.2 (SEM) at a week post immunization. Seven animals had a swelling with a maximum diameter of more than 4 cm. The swelling was in the majority of cases not painful and resolved to a small, apparently inert, nodule of approximately 1 cm diameter in the course of 3 weeks.

### 3.3 Fecal Culture Results

The results from the fecal culture tests are summarized in FIG. 1. Exponential increase of the number of fecal positive animals was observed during the first 210 days following experimental infection. At that point in time 12 of 28 animals (43%) had been fecal culture positive at least once, with a total cumulative fecal culture score per time point up to 32 at time point day 210. Following vaccination a sharp drop in fecal excretion of bacteria was observed. The total cumulative fecal culture score did not become higher than 12 during the remainder of the experiment, indicating a reduction in shedding of 62.5% following a single vaccination.

### 3.4 Tuberculin Skin Testing (FIG. 2)

As depicted in FIG. 2, none of the animals reacted positive in the comparative skin test when tested 8 weeks after vaccination with the Hsp70 subunit vaccine. In general fecal culture positive animals had a higher avian reaction compared to fecal culture negative animals, and in combination with low bovine tuberculin reaction indicative of the paratuberculosis infection.

### 3.4 IFN-γ Elispot (FIG. 3)

When comparing the pre-vaccination and the post-vaccination (30 days post vaccination) a significant IFN-γ response to PPD-P was found both pre and post vaccination compared to unstimulated control samples (student t-test, p<0.05). However neither before nor after vaccination a positive response to the Hsp70 immunogen was observed in the 28 animals, irrespective of their fecal culture status.

### 3.5 Lymphocyte Stimulation Test (FIG. 3)

An increased response to the Hsp70 immunogen was observed in the proliferation assay post vaccination, however this was only detected in fecal culture positive animals.

### 3.6 Serological Responses (FIG. 4)

Both in fecal culture positive animals and in fecal culture negative animals a clear antibody response was measured post vaccination. The antibody response was most prominent for the IgG1 isotype. A typical spike response shortly following vaccination was observed for IgG2. For IgM the fecal culture status did induce the highest response difference related to infection status. Fecal culture positive animals showed higher and more prolonged IgM response as compared to fecal culture negative animals. Serum IgA responses were generally low.

The tuberculin skin test procedure did not induce an antibody response to Hsp70 protein with any of the isotypes tested.

### 3.7 Serological Responses (FIG. 5)

Both in fecal culture positive animals and in fecal culture negative animals a clear antibody response to the Hsp70 N-terminus protein fragment (RBS70) was measured post vaccination. The antibody response was most prominent for the IgG1 isotype. A typical spike response shortly following vaccination was observed for IgG2. For IgM the fecal culture status did induce the highest response difference related to infection status. Fecal culture positive animals showed higher and more prolonged IgM response as compared to fecal culture negative animals. Serum IgA responses were generally low.

The tuberculin skin test procedure did not induce an antibody response to RBS70 protein with any of the isotypes tested.

Conclusion: it has been demonstrated that therapeutic vaccination of cattle with a Hsp70/DDA vaccine significantly reduces shedding of MAP in the feces which in turn may reduce transmission of infection, has little direct and long term side-effects, does not interfere with current tuberculosis diagnostics and enables differentiation between vaccinated and infected animals and as such may contribute to the paratuberculosis eradication strategies.

### LEGEND TO THE FIGURES

FIG. 1.

In panel A the fecal culture results from all individual animals is depicted for the 13 time points tested using the quantification as outlined in table 1. No data was acquired on time point 336 due to a technical failure. Animals were vaccinated with the Hsp70 subunit vaccine at day 210. Animal 1373 died at day 220. The total fecal culture score is depicted in panel B where the fecal culture scores were summed per time point as measure for total excretion.

FIG. 2.

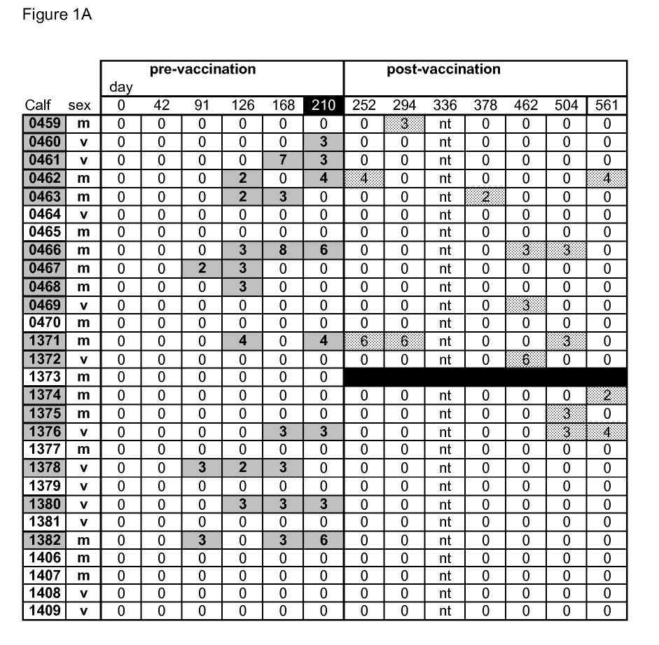

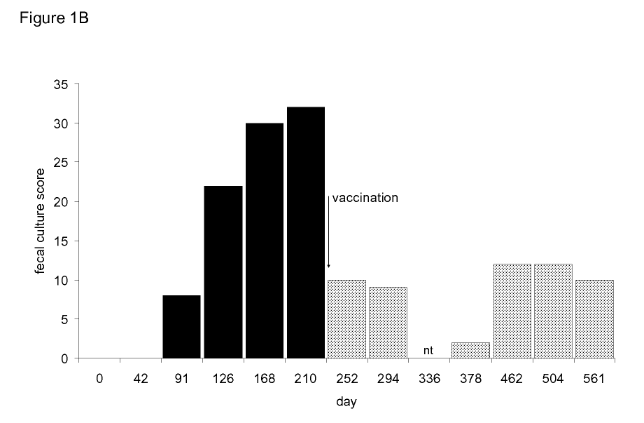

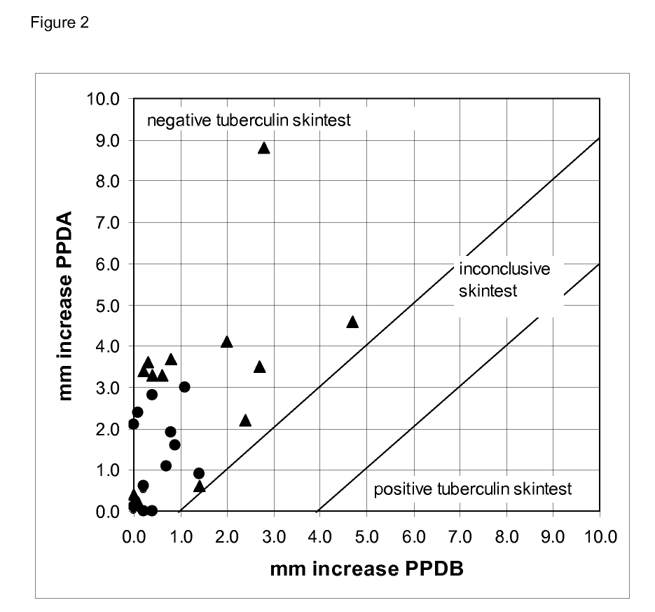

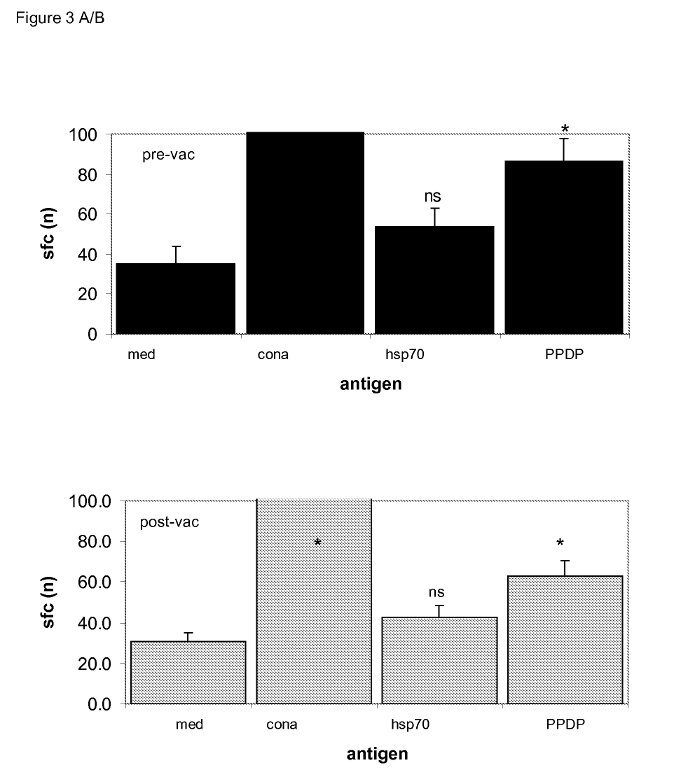

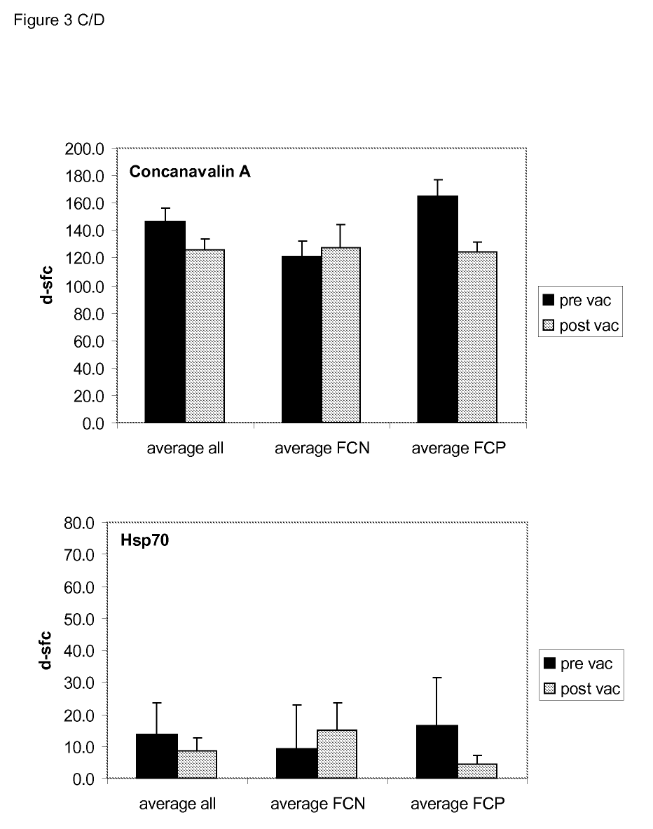

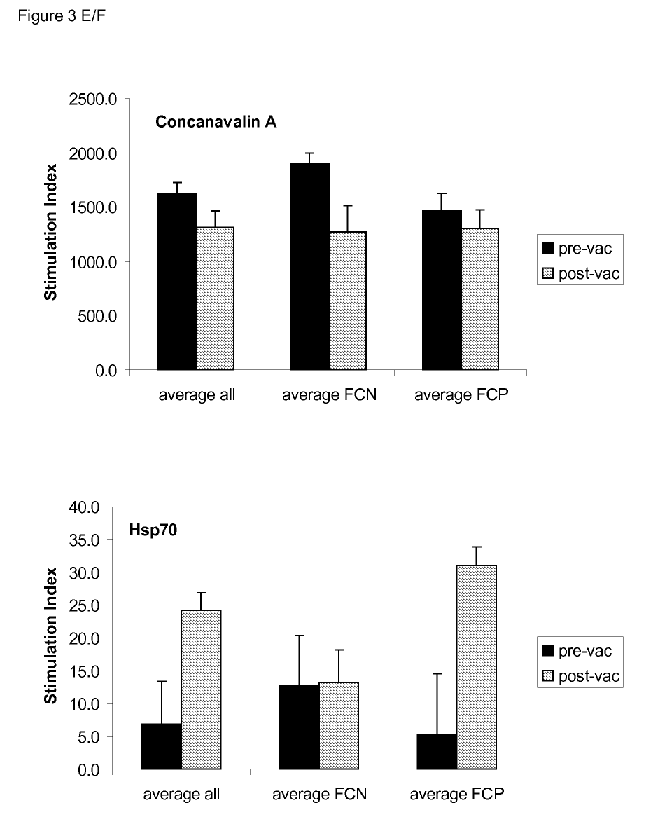

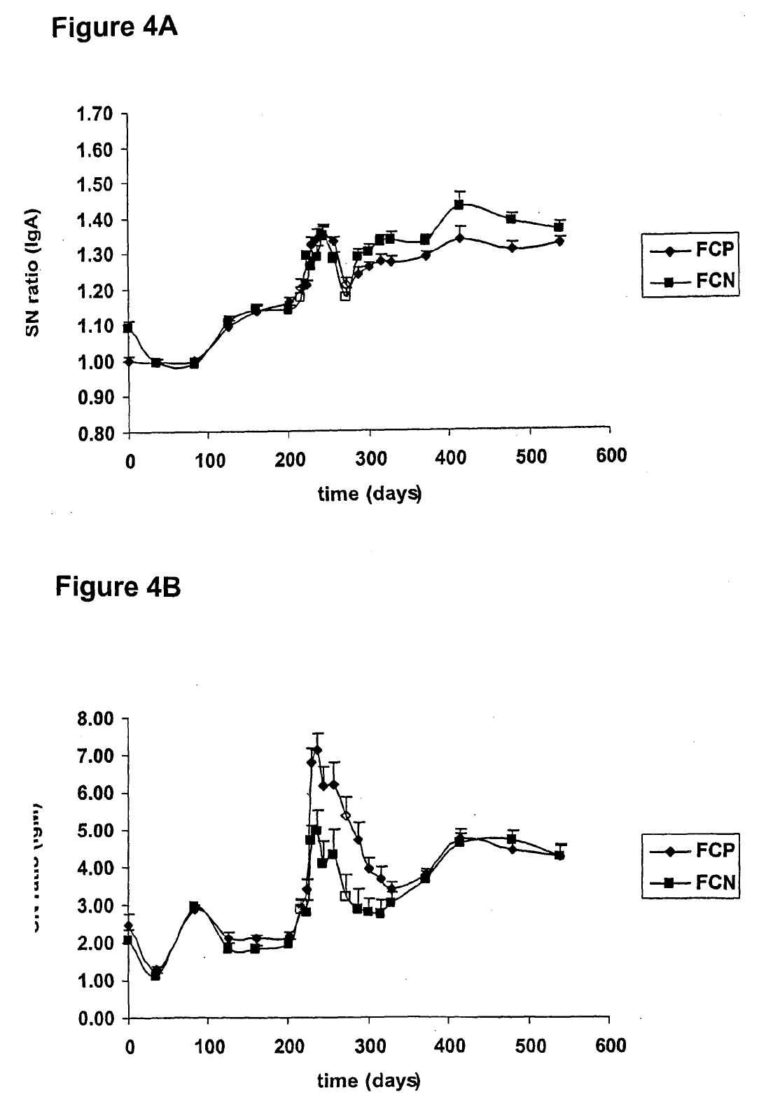

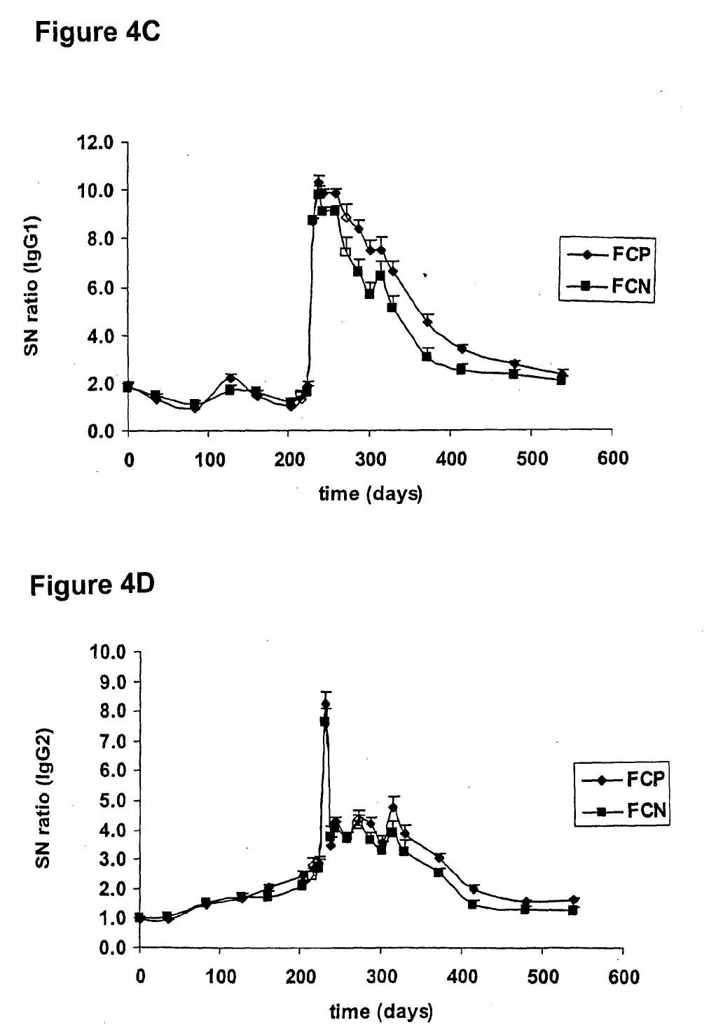

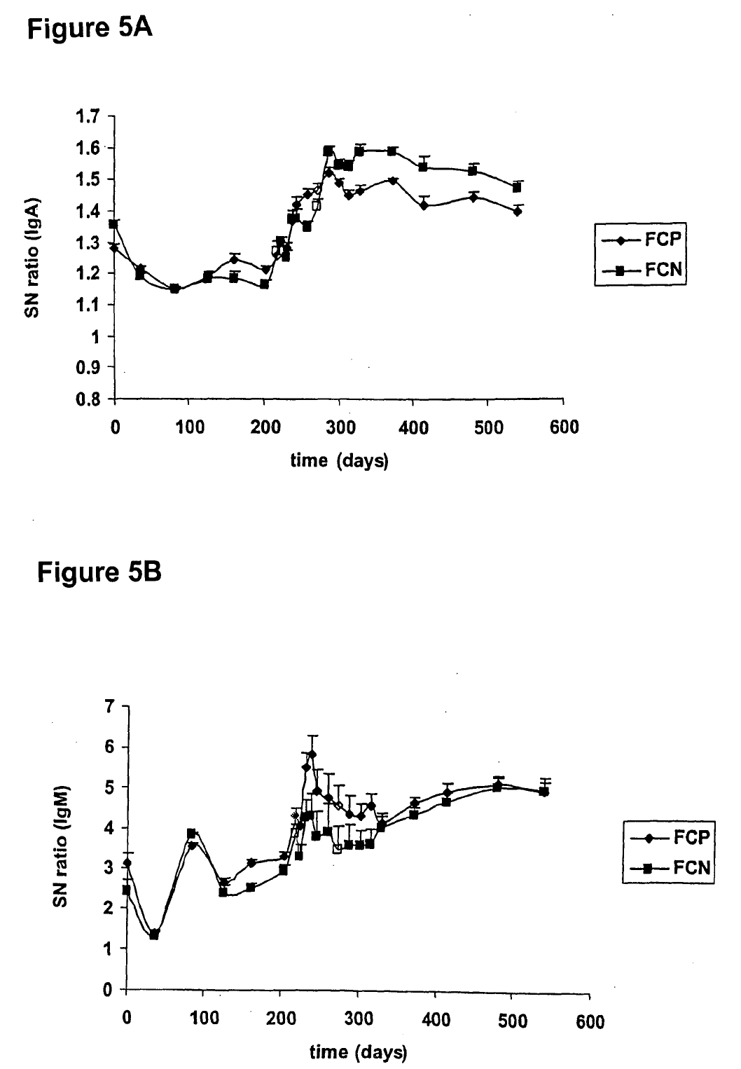

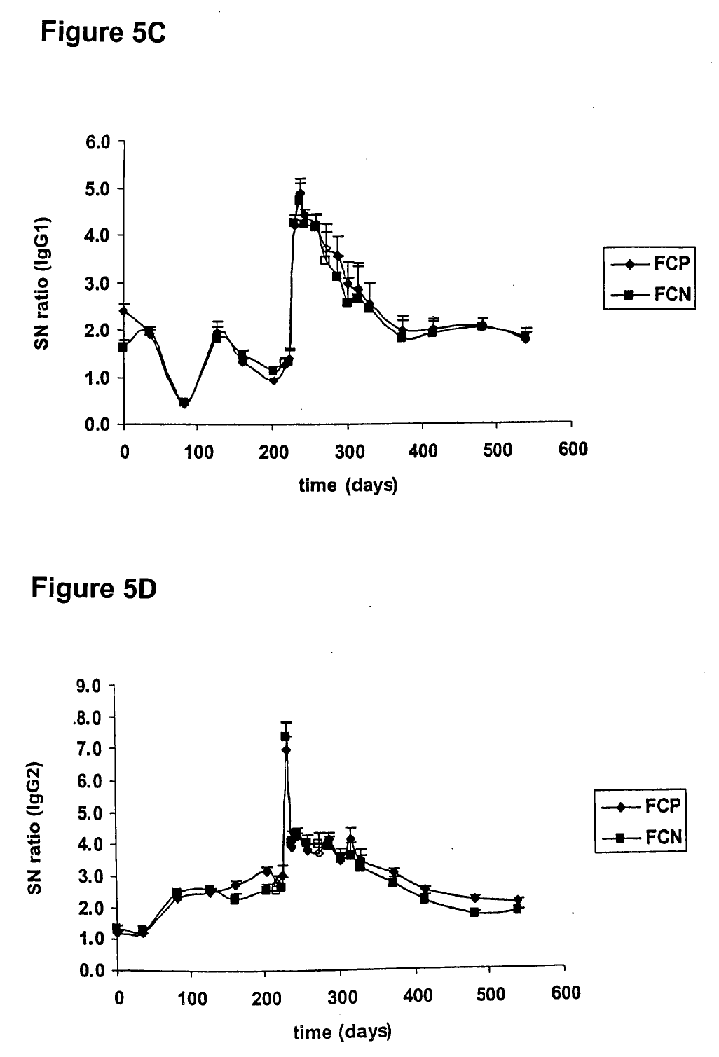

The tuberculin skin-fold reaction is depicted for all individual animals. Skin test data from 28 calves experimentally infected with MAP in the first month of life, and vaccinated with MAP Hsp70 eight weeks prior to the comparative skin test. The circles indicate calves that were fecal culture positive at least once in 6 tests before vaccination, the triangles indicate calves that were fecal culture negative. All animals tested negative in the comparative tuberculosis skin test. (Criteria according to EU guidelines for the intradermal comparative test for the establishment and maintenance of officially tuberculosis-free herd status)

FIG. 3.

The production of IFN-γ in the IFN-γ Elispot assay is expressed as the number of spot forming cells (sfc)/2.10⁵ PBMC. Antigens tested are the medium control, concanavalin A (positive), Hsp70 protein and PPDP. Average sfc results+SEM from cells from all animals pre vaccination (panel A) and 30 days post vaccination (panel B) are presented.

The production of IFN-γ in the IFN-γ Elispot assay is expressed as Δ-sfc; the number of spot forming cells (sfc)/2.10⁵ PBMC corrected for the medium control. Antigens tested are concanavalin A (positive control) (panel C), and Hsp70 protein (panel D). Average Δ-sfc results+SEM from cells from all animals, fecal culture negative animals and fecal culture positive animals are presented.

The proliferative response of PBMC pre and 30 days post vaccination are presented as stimulation index (SI) for positive control concanavalin A (panel E) and the Hsp70 protein (panel F). Average results+SEM from cells from all animals, fecal culture negative animals and fecal culture positive animals are presented.

FIG. 4.

The Hsp70 specific antibody-isotype response is depicted. Time of vaccination is at 210 days (open square), and tuberculin skin test is done after 266 days (open square). Results are average serum to negative (SN) ratio+SEM for fecal culture positive (diamonds) and negative animals (squares) separate. Figure A depicts the IgA response; figure B the IgM response; Figure C the IgG1 response; Figure D the IgG2 response.

FIG. 5.

The Hsp70 N-terminus fragment (RBS70) specific antibody-isotype response is depicted. Time of vaccination (open square, 210 days), and tuberculin skin test (open square, 266 days) are indicated. Results are average serum to negative (SN) ratio+SEM for fecal culture positive (diamonds) and negative animals (squares) separate. Figure A depicts the IgA response; figure B the IgM response; Figure C the IgG1 response; Figure D the IgG2 response.

1. A vaccine for treating animals infected with Mycobacterium comprising an immunologically effective amount of mycobacterial Hsp70 protein.

2. The vaccine according to claim 1, wherein the vaccine is for therapeutic application in humans.

3. The vaccine according to claim 1, wherein the vaccine is for therapeutic application in ruminants.

4. The vaccine according to claim 1, wherein the vaccine is for therapeutic application in cats or dogs.

5. The vaccine according to claim 1, wherein the mycobacterial Hsp7(protein is the Hsp70 protein of Mycobacterium avium.

6. The vaccine according to claim 5, wherein the mycobacterial Hsp70 protein is the Hsp70 protein of Mycobacterium avium ssp. paratuberculosis.

7. The vaccine according to claim 1, wherein the mycobacterial Hsp70 protein is the Hsp70 protein of Mycobacterium tuberculosis.

8. The vaccine according to claim 1, wherein the mycobacterial Hsp70 protein is the Hsp70 protein of Mycobacterium bovis.

9. A live recombinant carrier encoding mycobacterial Hsp70 or an immunogenic fragment thereof, under the control of a functionally linked promoter.

10. A host cell comprising a gene or a fragment thereof encoding mycobacterial Hsp70 or an immunogenic fragment thereof, under the control of a functionally linked promoter.

11. The vaccine according to claim 1, comprising an additional pathogenic microorganism or virus, antigenic material thereof or genetic material encoding said antigenic material.

12. The vaccine according to claim 11, wherein said additional pathogenic microorganism or virus is selected from the group of Bovine Herpesvirus, bovine Viral Diarrhoea virus, Parainfluenza type 3 virus, Bovine Paramyxovirus, Foot and Mouth Disease virus, Bovine Respiratory Syncytial Virus, porcine circo virus, porcine respiratory reproductive syndrome virus, another Mycobacterium spp, a Mycoplasma spp., Pasteurella haemolytica, Staphylococcus aureus, Escherichia coli, Leptospira spp., Staphylococcus uberis, Theileria parva, Theileria annulata, Babesia bovis, Babesia bigemina, Babesia major, Trypanosoma species, Anaplasma marginate, Anaplasma centrale and Neospora caninum.

13. The vaccine according to claim 1, additionally comprising an adjuvant.

14. The vaccine according to claim 13, wherein the adjuvant is DDA.

15. The vaccine according claim 1, wherein said vaccine is in a freeze-dried form.

16. A method for treating Mycobacterium infection in a mammal comprising administering a therapeutically effective amount of the vaccine according to claim 1.

17. The method of claim 16, wherein the mammal has been shedding Mycobacteria prior to treatment, whereby after administration of the vaccine shedding is reduced.

18. The method of claim 16, wherein the Mycobacterial Hsp70 protein is the Hsp 70 protein of Mycobacterium avium.
